(19) United States
(12) Patent Application Publication
(10) Pub. No.: US 2007/0089216 A1
(43) Pub. Date: Apr. 26, 2007

(54) Welding helmet

(76) Inventor: Charles D. Walkden, Homer, AK (US)

Correspondence Address: Alfred F. Hoyte, Esq., 7734 16th Street, N.W., Washington, DC, 20012, US

(21) Appl. No.: 11/398,320

(22) Filed: Apr. 6, 2006

Related U.S. Application Data
(60) Provisional application No. 60/728,767, filed on Oct. 21, 2005.

(51) Int. Cl. A61F 9/06

(52) U.S. Cl. 2/8.7

(57) ABSTRACT

A combined welding helmet apparatus is provided. The apparatus allows for several features to be incorporated into a single helmet. Particularly, several face and eye protection elements are incorporated into a single helmet. An integrated internal mechanism allows for selected elements to be functionally positioned while non-selected elements are unobtrusively positioned to a non-functional position. At least three face and eye protection elements are combined in the helmet; a dark filtered welding lens, a lighter filtered lens, and a clear safety plate.

### BACKGROUND OF THE INVENTION

The present invention relates to protective gear for welders. More particularly, it relates to an improved welding helmet having multiple viewing lenses.

### STATEMENT OF THE PRIOR ART

Welding helmets having lenses to attenuate light emitting from the welding torch are well known. These helmets generally are available in two types, a first type having an ambient light responsive liquid crystal lens, and a second type having a manually positionable, visually opaque lens.

The liquid crystal lens assembly comprises a layer of nematic liquid crystal material sandwiched between opposing parallel plates coated with transparent conductive films. The faces of the plates in contact with the liquid crystal material are treated so as to effect a twisted nematic structure therein which will rotate polarized light passing through the light shutter through 90.degree. Polarizers are disposed on opposite sides of the plates such that by applying an electrical field across the transparent conductive films, the opacity of the liquid crystal light shutter can be changed. In order to render the lens light-transmitting, an electric field must be established across the conductive films. Thus, in the event of power failure, the lens will, theoretically, automatically assume an opaque condition and protect the eyes of the welder, with the obvious drawback that if the welder is not engaged in welding, he/she will be briefly blinded which, in a construction setting could lead to accidents.

The other type of helmet has a single manually positionable opaque lens element, which is usually pivotable by the movement of the welders head into an operational position. These helmets are structured to position only a single lens element in front of a viewing aperture which may or may not be shielded by a transparent plate.

Attempts have been made to provide for a number of interchangeable lenses for a welding helmet, but the helmets produced have had drawbacks. For example, U.S. Pat. No. 6,151,711 issued to Edwards on Nov. 28, 2000 discloses a welding helmet which includes a removable lens cartridge assembly which is slidingly received by a slot at the front of the welding helmet. The lens cartridge assembly includes a picture frame slot into which a face lens may be friction fit by insertion from the side. Additional filters and lenses may be held in place within the cartridge by a removable frame assembly. Edwards suffers from the drawback that there is no way to store the additional lenses and thus they are subject to being misplaced. Also, as the lens must be slid into position to cover the viewing aperture, improper positioning of the lens could result in a small gap between the lens frame and the helmet body, with the possibility of injury to the welder.

### SUMMARY OF THE INVENTION

The present invention contemplates a combined welding helmet apparatus is provided. The apparatus allows for several features to be incorporated into a single helmet. Particularly, several face and eye protection elements are incorporated into a single helmet. An integrated internal mechanism allows for selected elements to be functionally positioned while non-selected elements are unobtrusively positioned to a non-functional position. At least three face and eye protection elements are combined in the helmet; a dark filtered welding lens, a lighter filtered lens, and a clear safety plate.

Accordingly, it is a principal object of the invention to provide a new and improved welding helmet having multiple viewing options.

It is another object of the invention to provide a new and improved welding helmet having multiple viewing options where all of the options are manually selectable.

It is another object of the invention to provide a new and improved welding helmet having easily accessible means for manually reconfiguring the helmet.

Finally, it is a general object of the invention to provide improved elements and arrangements thereof in an apparatus for the purposes described which is fully effective in accomplishing its intended purpose.

These and other objects of the present invention will become readily apparent upon further review of the following specification and drawings.

### BRIEF DESCRIPTION OF THE DRAWINGS

Various other objects, features, and attendant advantages of the present invention will become more fully appreciated as the same becomes better understood when considered in conjunction with the accompanying drawings, in which like reference characters designate the same or similar parts throughout the several views, and wherein:

FIG. 1 shows a plan view of the welding helmet of the present invention.

FIG. 2 shows a side view, partly in section, of the welding helmet of the present invention positioned on a user's head.

FIG. 3 shows a side sectional view of the welding helmet with the plasma cutting lens in the operational position.

FIG. 4 shows a side sectional view of the welding helmet with the welding lens in the operational position.

FIG. 5 shows a side sectional view of the welding helmet arranged to allow for full light transmissivity.

### DETAILED DESCRIPTION

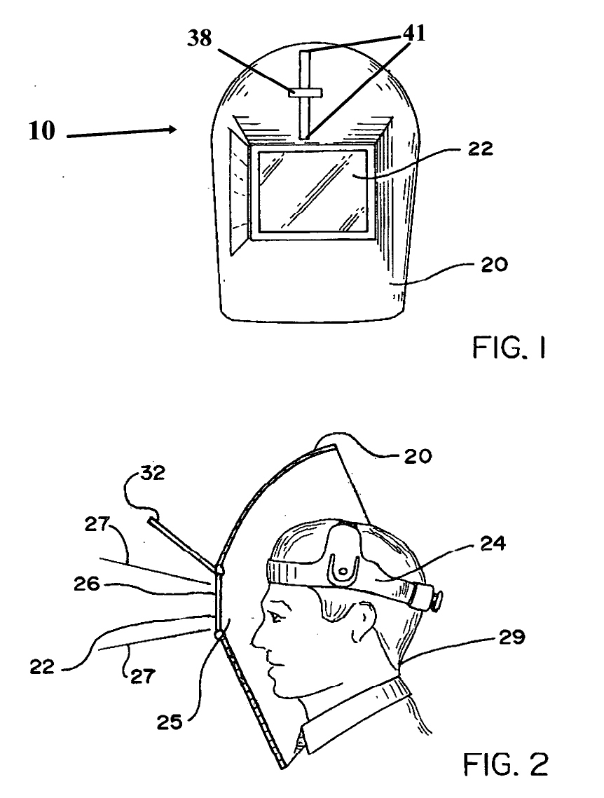

Referring now to FIGS. 1-5, the welding helmet of the present invention, generally indicated by the numeral 10, is shown. It can be seen that the welding helmet 10 comprises a relatively large, visually opaque main body 20 having a central viewing aperture 22 formed in the front portion thereof. A standard attachment means 24 is used to secure the main body, and once attached to a user's head 29, allows the main body 20 to be pivoted to shield the user's face. The attachment means 24 is secured to the main body 20 in journaled relation by opposing pivots 23.

Selectively positioned proximate the central viewing aperture 22 are at least two lenses for reducing the amount of light transmitted through the viewing aperture. A safety plate or window 26, which is transparent to allow all light to pass therethrough covers the aperture 22 and serves to deflect debris, sparks, and the like as may be produced by the welding process, or any sawing and grinding processes related-thereto. The aperture 22 and window 26 are positioned perpendicular to the viewing axis 25 to allow for viewing a workpiece therethrough, the workpiece visible through a viewing angle approximately as shown by lines 27. Hingedly connected to the front panel 31 of the helmet main body 20 is a welding lens 30, which may be a piece of welder's glass such as Crook's Glass which is especially formulated to reduce the transmissivity through the window 26 and also to absorb substantially all ultraviolet and most of the blue light from the work. The lens 30 may be secured to the pivoting lens frame 32 by any conventional means, and may be removably secured to allow for periodic replacements. It can be seen that the lens 30 can be selectively positioned in front of viewing aperture 22 to allow for welding.

As is well known in the art, welding helmets are useful as protective gear for a variety of tasks besides welding. For example, a welding helmet could be used for any sawing or grinding process as implied above, especially when metals are being worked as flying debris therefrom is heavier and therefore more dangerous than sawdust. To that end, welding helmets having the so-called light responsive lenses have been developed as noted above. These lenses are designed to respond quickly to changes in the ambient light intensity, but require a constant supply of electric current to do so. The present invention contemplates a welders helmet having a sufficient number of auxiliary lenses so that a lens for any task may be manually selected. At present, there are three types of tasks which are facilitated by the use of protective head gear, the welding helmet 10 of the invention therefore has at least three discrete configurations, and means for manually switching to a desired configuration. As previously mentioned, grinding and sawing processes may be performed by viewing the workpiece through window 26 with no other lenses in the user's line of sight. For welding operations, the lens 30 may be positioned to be in the user's line of sight as also discussed above.

A second task commonly performed by welders is the cutting of metals and the like with a plasma torch cutting tool. Plasma arc torches, also known as electric arc torches, are commonly used for cutting, marking, gouging, and welding metal workpieces by directing a high energy plasma stream consisting of ionized gas particles toward the workpiece. These types of torches create a fairly bright arc, but one that does not require a visually opaque lens. A shaded or tinted lens is commonly used for this task as is known by those of skill in the art. To that end, an additional lens 36 is slidably disposed interiorly of the main body 20. The lens frame 34 for lens 36 is attached to longitudinally extending connecting member 37 by screws or the like which is connected to actuator tab or actuator 38 which can be reciprocally slid within a slot 39 formed in the main body 20, as indicated by arrows A and B. The forward travel of the actuator 38 is limited by the length of the slot 39, the ends of which form mutually opposed abutment surfaces 41. By moving the actuator briskly 38, the user will hear an audible click indicating that the lens 36 is in the proper position, the click indicating abutment of the actuator with an abutment surface. Other means as would be apparent to one of skill in the art may be used to provide an indication that the actuator has reached the end of its travel. As with the welding lens 30, frame 34 is preferably configured to allow for periodic replacement of the lens 30.

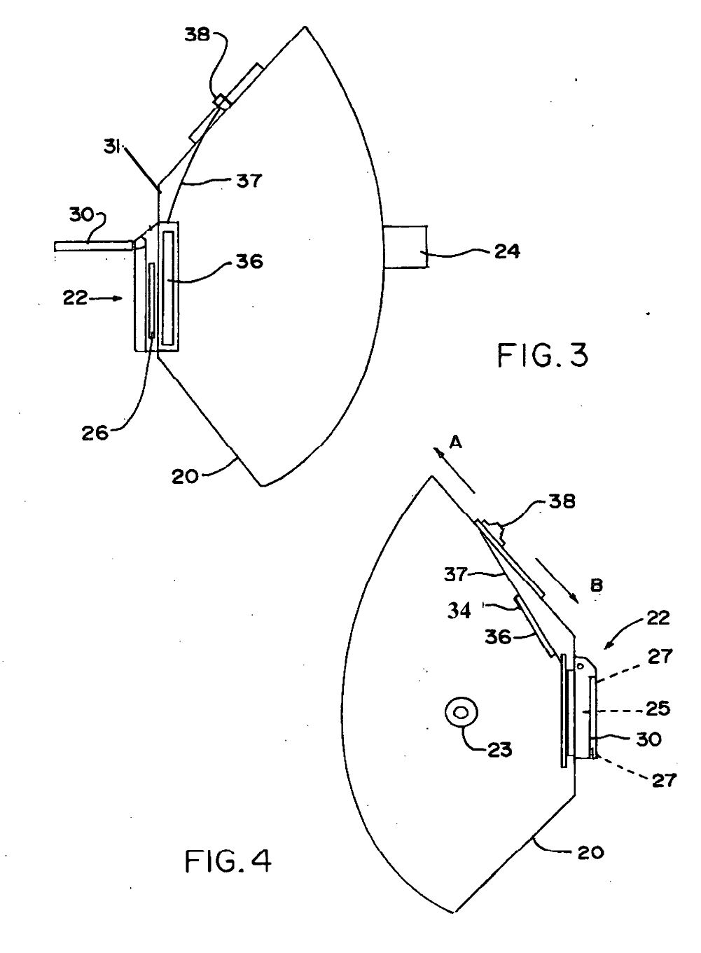

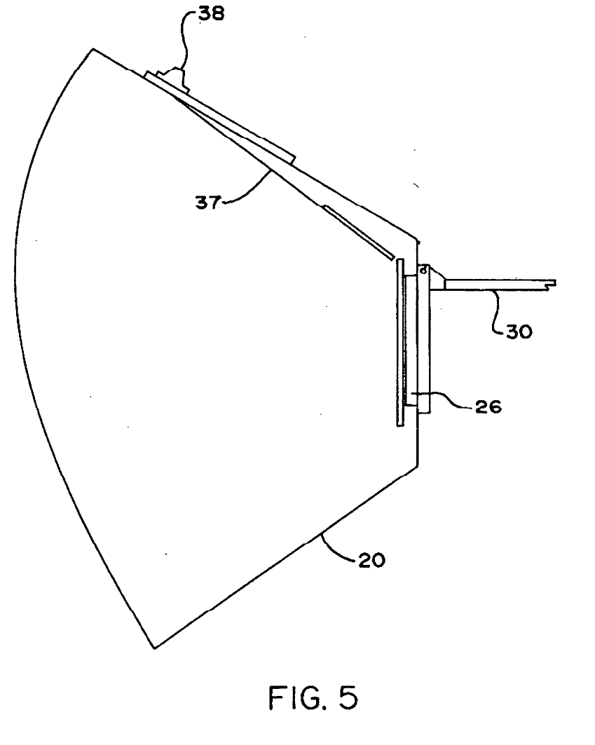

In operation, for grinding and sawing operations, the helmet 10 is configured as shown in FIG. 5, by pivoting the welding lens 30 to the horizontal position, and sliding the actuator 38 for lens 36 in the direction of arrow A until it reaches its limit of travel. For welding operations, the welding lens 30 is pivoted or flipped down into the vertical position as shown in FIG. 4, thereby entering the user's line of vision and completely obscuring window 26. Finally, for plasma torch cutting, welding lens 30 is flipped into the horizontal position, and lens 36 is slid in the direction of arrow B so that the helmet 10 is configured as shown in FIG. 3

From the foregoing description, one skilled in the art can easily ascertain the essential characteristics of this invention and, without departing from the spirit and scope thereof, can make various changes and modifications of the invention to adapt it to various usages and conditions.

It is to be understood that the present invention is not limited to the sole embodiment described above, but encompasses any and all embodiments within the scope of the following claims:

What is claimed is:

1. A welding helmet assembly comprising:
a visually opaque main body having a viewing aperture formed in a front panel;
a transparent window secured within said aperture;
a first lens assembly hingedly attached to said front panel and selectively positionable proximate to said transparent window for affecting ambient light radiating therethrough;
a second lens assembly slidably positioned interiorly of said main body and selectively positionable proximate to said transparent window for affecting ambient light radiating therethrough.

2. The helmet assembly of claim 1 wherein said first lens assembly removably contains a visually opaque welding lens.

3. The helmet assembly of claim 1 wherein said second lens assembly removably contains a tinted lens.

4. The helmet assembly of claim 1 wherein said second lens assembly includes an actuator slidably disposed within said main body, said actuator including means for providing an audible indication when said tinted lens is positioned proximate said transparent window.

5. A welding helmet assembly comprising:
a visually opaque main body having a viewing aperture formed in a front panel;
a transparent window secured within said aperture;
a first lens assembly hingedly attached to said front panel and selectively positionable proximate to said transparent window for affecting ambient light radiating therethrough;
a second lens assembly slidably positioned interiorly of said main body and selectively positionable proximate to said transparent window for affecting ambient light radiating therethrough;
said first and second lens assemblies positionable in mutually independent fashion.

6. The helmet assembly of claim 5 wherein said first lens assembly removably contains a visually opaque welding lens.

7. The helmet assembly of claim 5 wherein said second lens assembly removably contains a tinted lens.

8. The helmet assembly of claim 5 wherein said second lens assembly includes an actuator slidably disposed within said main body, said actuator including means for providing an audible indication when said tinted lens is positioned proximate said transparent window.
